(19) United States
(12) Patent Application Publication
(10) Pub. No.: US 2018/0070967 A1
(43) Pub. Date: Mar. 15, 2018

(54) AIR BUBBLE REMOVAL LITHOTRIPSY ASSEMBLY AND METHOD

(71) Applicant: M. Fazni Aziz, Bloomington, IN (US)

(72) Inventor: M. Fazni Aziz, Bloomington, IN (US)

(21) Appl. No.: 15/261,150

(22) Filed: Sep. 9, 2016

(51) Int. Cl. A61B 17/22

(52) U.S. Cl. CPC A61B 17/22004; A61B 2017/22025; A61B 2017/22014

(57) ABSTRACT

A lithotripsy assembly includes a target positioned inside a body that has an external surface. A lithotripsy dry head shock wave transducer is aimed at the target. A gel enclosure is defined at least partially by the body and the lithotripsy transducer. A volume of acoustic coupling fluid is positioned in the gel enclosure, which is fluidly connected to a vacuum pump. The vacuum pump draws air bubbles out of the gel enclosure to reduce attenuation of shock waves.

### TECHNICAL FIELD

The present disclosure relates generally to devices and methods for performing lithotripsy, and more particularly to reducing shock wave attenuation by removing air bubbles from acoustic coupling fluid.

### BACKGROUND

Lithotripsy is known as a procedure by which shock waves are transmitted into a body to cause a kidney stone to fracture into smaller stones that may more easily be passed. The relatively recent introduction of dry treatment heads has potentially eliminated the need to immerse a patient in a water bath in order to successfully transmit a shock wave to the target stone. Dry treatment heads require some acoustic coupling fluid to facilitate transmission of the shock wave through the coupling fluid and into the body to the target stone. Some researchers have suggested that dry head lithotripsy strategies can suffer performance shortcomings due to the presence of air bubbles in the acoustic coupling fluid.

The present disclosure is directed toward one or more of the problems set forth above.

### SUMMARY

In one aspect, a lithotripsy assembly includes a target positioned inside a body that has an external surface. A lithotripsy dry head shock wave transducer is aimed at the target. A gel enclosure is defined at least partially by the body and the lithotripsy transducer. A volume of acoustic coupling fluid is positioned in the gel enclosure. A vacuum pump is fluidly connected to the gel enclosure to draw air bubbles out of the gel enclosure toward the vacuum pump.

In another aspect, an air bubble removal apparatus for use with a lithotriptor includes a double sided cuff with an annular wall that terminates on one side with a first annular seal and on an opposite side with a second annular seal. The first annular seal is for contacting a lithotripsy dry head shock wave transducer, and the second annular seal is for contacting a body. The double sided cuff has an annular wall partially defining a gel enclosure. A pump is fluidly connected to the gel enclosure through at least one port that opens through the annular wall. An acoustic coupling fluid reservoir is fluidly connected to the gel enclosure through the at least one port.

In still another aspect, a method of operating a lithotripsy assembly includes positioning an acoustic coupling fluid in a gel enclosure between a lithotripsy dry head shock wave transducer and a body. Air bubbles are removed from the gel enclosure by fluidly connecting the gel enclosure to a vacuum pump. A shock wave is transmitted from the lithotripsy dry head shock wave transducer through the acoustic coupling fluid to a target stone in the body.

### BRIEF DESCRIPTION OF THE DRAWING

FIG. 1 is a schematic perspective view a lithotripsy assembly according to one aspect of the present disclosure;

FIG. 2 is a sectioned view through the body, cuff and transducer during shock wave transmission for the lithotripsy assembly of FIG. 1;

FIG. 3 is a schematic perspective view of a double sided cuff for an air bubble removal apparatus according to another aspect of the present disclosure;

FIG. 4 is a schematic view showing different variations of an air bubble removal apparatus according to the present disclosure;

FIG. 5 is a sectioned view through the gel enclosure with air bubbles present;

FIG. 6 is a sectioned view similar to FIG. 5 aspect after actuation of the vacuum pump according to the present disclosure; and

FIG. 7 is a sectioned view similar to FIGS. 5 and 6 except after air bubbles have been evacuated from the gel enclosure.

### DETAILED DESCRIPTION

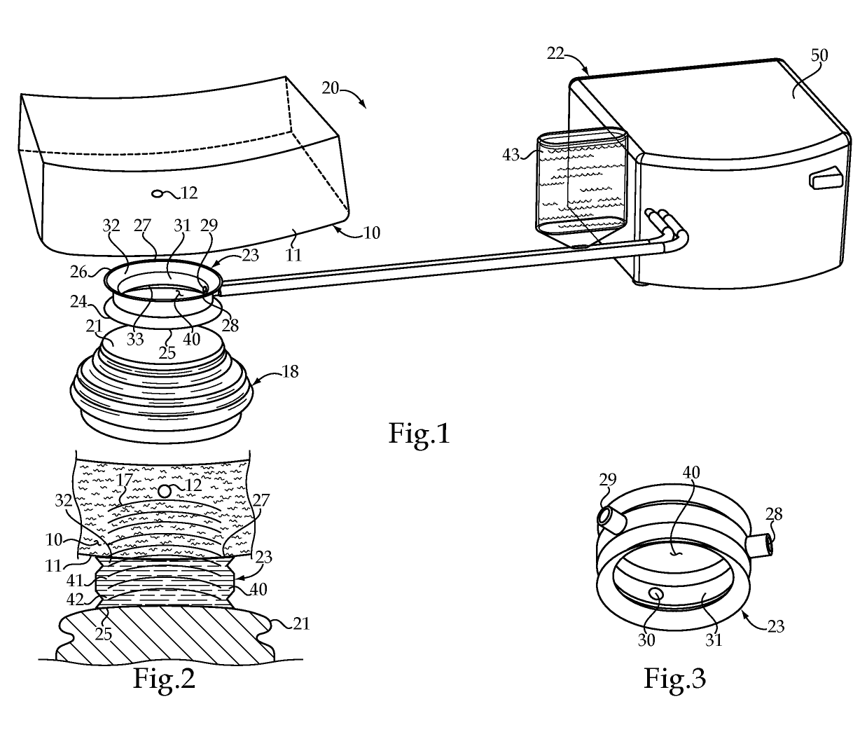

Referring initially to FIGS. 1 and 2, a lithotripsy assembly 20 includes a body 10 that has an external surface 11 and includes a target 12 positioned in the body 10. Body 10 may be a live patient, or may be an artificial body used for purposes of teaching or demonstration. Target 12 could be a target stone if the body is a live patient, or could be a target for purposes of teaching or demonstration. A lithotriptor 18 includes a lithotripsy dry head shock wave transducer 21 that is aimed at the target 12. A gel enclosure 40 is defined at least partially by the body 10 and the lithotripsy transducer 21, as best shown in FIG. 2. A volume 41 of acoustic coupling fluid 42 is positioned in the gel enclosure 40. A vacuum pump 50 is fluidly connected to the gel enclosure 40. The vacuum pump 50 draws air bubbles out of the gel enclosure 40 toward the vacuum pump to decrease attenuation of a shock wave 17 to more effectively fracture a target stone 12. An acoustic coupling fluid reservoir 43 may be fluidly connected to the gel enclosure 40. The acoustic coupling fluid 42 may be an oil, water or gel or other acoustic coupling fluid known in the art.

FIG. 1 can also be thought of as showing an air bubble removal apparatus 22 for use with a lithtriptor 18. Apparatus 22 includes a double sided cuff 23 that includes an annular wall 31 that terminates on one side 24 with a first annular seal 25 for contacting the lithotripsy dry head shock wave transducer 21. The double sided cuff 23 also terminates on an opposite side 26 with a second annular seal 27 for contacting the external surface 11 of body 10. The annular wall 31 partially defines the gel enclosure 40. A pump, which in this illustrated embodiment is a vacuum pump 50, is fluidly connected to the gel enclosure 40 through a port 28 that opens through the annular wall 31. The acoustic coupling fluid reservoir 43 is fluidly connected to the gel enclosure 40 through a second port 29. When vacuum pump 50 is actuated, acoustic coupling fluid 43 that is free of air bubbles moves toward and into gel enclosure 40 while air bubbles are drawn from gel enclosure 40 toward vacuum pump 50.

Referring now to FIG. 3, a double sided cuff 23 according to another embodiment of the present disclosure is shown with identical numbers for the same named features. In this embodiment, ports 28 and 29 open at spread apart locations approximately 120° apart through the annular wall 31 of double sided cuff 23. By locating the ports 28 and 29 somewhat opposite of one another, the pump 50 may more effectively and efficiently move bubbles out of the gel enclosure 40 that is partially defined by the annular wall 31. FIG. 3 is also of interest for teaching and showing a third port 30 that is opens through annular wall 31 equally spaced from ports 28 and 29. Depending upon a variety of factors in most effectively moving air bubbles, third port 30 may be fluidly connected to one of the vacuum pump 50 and the acoustic coupling fluid reservoir 43. Thus, depending upon design choice, more than one of the ports 28, 29, 30 maybe fluidly connected to either the pump 50 or the reservoir 43. Those skilled in the art will appreciate that the present disclosure contemplates any number of ports including a only a single port without departing from the present disclosure.

As best shown in FIGS. 1 and 2, the gel enclosure 40 may include a frustoconical surface 32 in contact with body 10, and a second frustoconical surface 33 that may be contact with the lithotripsy dry head shock wave transducer 21. Frustoconical surfaces 32 and 33 may be portions of the double sided cuff 23 and help facilitate seals on the respective external surface 11 of body 10 and transducer 21. The gel enclosure 40 is partially defined by the annular wall 31, which includes frustoconical surfaces 32 and 33. Referring briefly to FIG. 5, the double sided cuff 23 may be equipped with an air bubble sensor 60, such as the optical sensor shown, to provide information for possible control of vacuum pump 50. For instance, pump 50 may be operated until air bubble sensor 60 detects a lack of air bubbles 61 in the acoustic coupling fluid 42 that is positioned in gel enclosure 40.

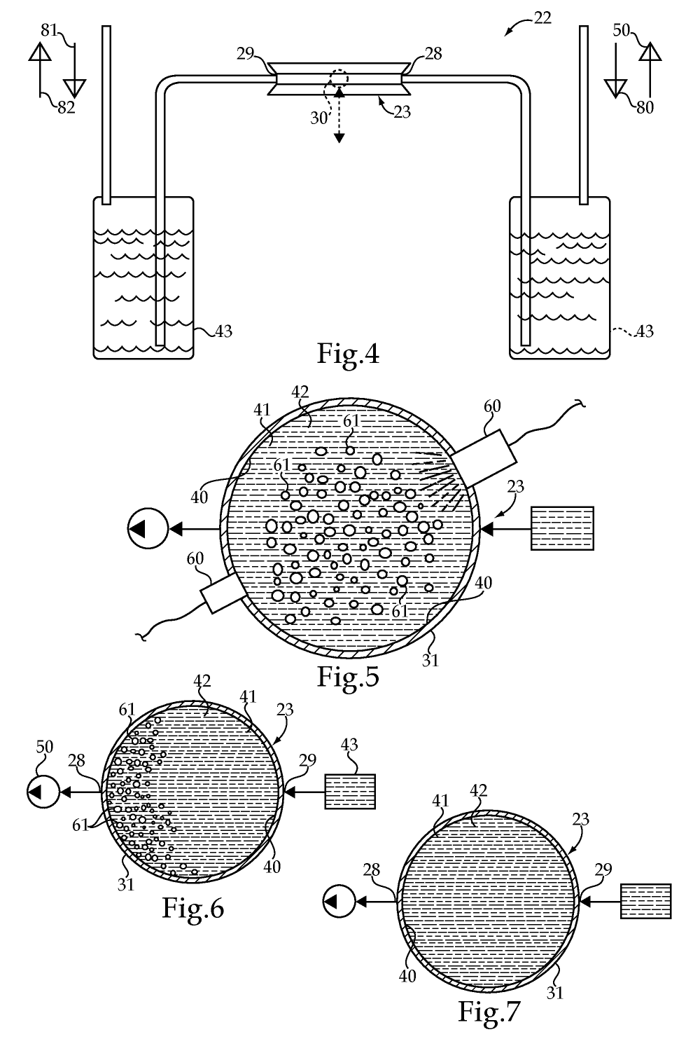

Referring now to FIG. 4, a schematic illustrates some variations on a air bubble removal apparatus 22 according to the present disclosure. For instance, the schematic shown in FIG. 4 can read upon the embodiment of FIG. 1 with the acoustic coupling fluid reservoir 43 fluidly connected to port 29 of double sided cuff 23, while port 28 is fluidly connected to vacuum pump 50. One alternative embodiment could be substituting a pressure pump 80 in place of vacuum pump 50, in which case the right hand side reservoir could be considered the acoustic coupling reservoir 43 as shown with a dashed leader line fluidly connected to the double side cuff 23 via a single port 28. Acoustic coupling fluid could flow and spill out of cuff 23 onto the body or transducer (not shown), or could exit cuff 23 at port 29 into a take up reservoir which would be located on the left. In still another embodiments, an additional pump may be utilized, such as for instance a second pressure pump 81 or a second vacuum pump 82, and possibly utilize a third port 30 with various fluid connections as would occur to someone with ordinary skill in the art to remove air bubbles from acoustic coupling fluid in double sided cuff 23.

### INDUSTRIAL APPLICABILITY

The present disclosure finds general applicability for use as a portion of a lithotripsy assembly, and more particularly to a strategy for removing air bubbles between a lithotripsy transducer and a body. The present disclosure finds specific applicability in association with lithotripsy dry head shock wave transducers.

In general, the procedure may be performed by first placing cuff 23 onto the lithotriptor dry head transducer 21. Next, the various tubing may be attached to the vacuum pump 50 and to the double sided cuff 23. The patient, or artificial body 10, may be oriented to a correct position with regard to the transducer 21. Next, the cycle of removing air bubbles is initiated by operation of vacuum pump 50. After the acoustic coupling fluid 42 is free of air bubbles, the lithotripsy procedure may be initiated by transmitting a shock wave into the body.

Referring now to all the Figs., a method of operating a lithotripsy assembly 20 is performed by positioning an acoustic coupling fluid 42 in a gel enclosure 40 between a lithotripsy dry head shock wave transducer 21 and body 10. Air bubbles 61 are removed from the gel enclosure 40 by fluidly connecting the gel enclosure 40 to the vacuum pump 50, as shown by the transition from FIG. 5 to FIG. 6. After the air bubbles are removed as shown in FIG. 7, the vacuum pump 50 may be switched off or remain operating. In any event, a shock wave 17 (FIG. 2) is transmitted from the lithotripsy dry head shock wave transducer 21 through the acoustic coupling fluid 42 to a target stone 12 in body 10. While the vacuum pump 50 is operating, acoustic coupling fluid 42 may be moved from a reservoir 43 toward the gel enclosure 40 responsive to operation of the vacuum pump 50. Those with ordinary skill in the art will appreciate that when vacuum pump 50 is in operation, and possibly after the vacuum pump 50 is turned off, provided that proper seals are made, pressure within gel enclosure 40 may be below atmospheric pressure.

Although testing may reveal that vacuum pump 50 can reliably move air bubbles 61 from the gel enclosure 40 in an open loop control fashion, such as by operating for a fixed duration on the order of maybe 10-60 seconds, the present disclosure also contemplates closed loop control. For instance, an optical bubble sensor 60 may be included on double sided cuff 23 to detect the presence of air bubbles in gel enclosure 40, and terminate operation of vacuum pump 50 or otherwise signal the lack of bubbles to an operator responsive to a lack of detection of air bubbles 61. Thus, the vacuum pump 50 may be operable to stop operation responsive to sensing an absence of air bubbles 61 in gel enclosure 40. Preferably, the gel enclosure becomes a closed volume by contacting a first annular seal 25 with the transducer 21, and a second annular seal of the double sided cuff 23 with body 10. As previously discussed, and as shown in sequence of FIGS. 5-7, air bubbles are moved in the gel enclosure through port 28 in annular wall 31 of double sided cuff 23 toward the vacuum pump 50. While this is occurring acoustic coupling fluid 42 may be moved into the gel enclosure 40 through a second port 29 in the annular wall 31 responsive to operation of the vacuum pump 50.

In practice, starting the vacuum pump 50 may help to serve to create a seal between the transducer 21 and the cuff 23, plus the body 10 and the cuff 23. Once the vacuum pump reaches a set point, which maybe measured by a built in pressure sensor (not shown), a valve may release gel or other acoustic coupling fluid from the reservoir 43. When the gel fills the double sided cuff 23, the pressure may be at or below atmospheric pressure. However, at this point the cuff will only be partially filled with gel. This cycle may be repeated a few times until the cuff 23 is filled with acoustic coupling fluid. Also, this could happen as a continuous process while the vacuum pump 50 is active. Most of the air bubbles may be removed from the gel prior to this procedure, so that the end of this process the gel and the cuff 23 will have a minimal amount of air bubbles. Next, the vacuum line may be used to reduce the volume inside the gel enclosure 40 to decrease the thickness of acoustic coupling gel 42 that the shock wave 17 must travel through.

Any time a vacuum is applied to the cuff 23, assuming that both sides are sealed, the patients skin may be pulled into the cuff 23, the cuff height will reduce, and the dry head of the lithotriptor transducer 21 will be sucked in. At this point, the pressure inside the cuff 23 may be lower than atmospheric pressure. Once the operator or control unit determines that the acoustic coupling fluid is free of air bubbles, the lithotripsy procedure may commence. Although the previous discussion shows that an air bubble sensor may be in the form of an optical air bubble sensor, those skilled in the art will appreciate that other features such as a camera, a light with a receiver, etc. may be substituted without leaving the intended scope of the present disclosure.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A lithotripsy assembly comprising:
a body having an external surface and including a target positioned inside the body;
a lithotripsy dry head shock wave transducer aimed at the target;
a gel enclosure defined at least partially by the body and the lithotripsy transducer;
a volume of acoustic coupling fluid positioned in the gel enclosure;
a vacuum pump fluidly connected to the gel enclosure; and
wherein the vacuum pump draws air bubbles out of the gel enclosure toward the vacuum pump.

2. The lithotripsy assembly of claim 1 including an acoustic coupling fluid reservoir fluidly connected to the gel enclosure; and
the vacuum pump draws acoustic coupling fluid from the reservoir toward the gel enclosure.

3. The lithotripsy assembly of claim 1 wherein the gel enclosure is defined partially by an annular wall;
the vacuum pump is fluidly connected to the gel enclosure through a port that opens through the annular wall.

4. The lithotripsy assembly of claim 1 wherein the gel enclosure is defined partially by an annular wall;
an acoustic coupling fluid reservoir is fluidly connected to the gel enclosure through a port that opens through the annular wall.

5. The lithotripsy assembly of claim 1 wherein the gel enclosure includes a frustoconical surface in contact with the body.

6. The lithotripsy assembly of claim 1 wherein the gel enclosure includes a frustoconical surface in contact with the lithotripsy dry head shock wave transducer.

7. The lithotripsy assembly of claim 1 including an air bubble sensor positioned to detect a presence of air bubbles in the acoustic coupling fluid in the gel enclosure.

8. The lithotripsy assembly of claim 1 wherein the gel enclosure is defined partially by an annular wall;
the vacuum pump is fluidly connected to the gel enclosure through a first port that opens through the annular wall; and
an acoustic coupling fluid reservoir fluidly connected to the gel enclosure through a second port that opens through the annular wall.

9. An air bubble removal apparatus for use with a lithotriptor comprising:
a double sided cuff having an annular wall that terminates on one side with a first annular seal for contacting a lithotripsy dry head shock wave transducer, and terminates on an opposite side with a second annular seal for contacting a body, and the annular wall partially defining a gel enclosure;
a pump fluidly connected to the gel enclosure through at least one port that opens through the annular wall; and
an acoustic coupling fluid reservoir fluidly connected to the gel enclosure through the at least one port.

10. The lithotriptor assembly of claim 9 including an air bubble sensor positioned to detect a presence of air bubbles in the acoustic coupling fluid in the gel enclosure.

11. The lithotriptor assembly of claim 9 wherein the pump is a vacuum pump.

12. The lithotriptor assembly of claim 9 wherein each of the first annular seal and the second annular seal includes a frustoconical surface.

13. A method of operating a lithotripsy assembly comprising the steps of:
positioning an acoustic coupling fluid in a gel enclosure between a lithotripsy dry head shock wave transducer and a body;
removing air bubbles from the gel enclosure by fluidly connecting the gel enclosure to a vacuum pump;
transmitting a shock wave from lithotripsy dry head shock wave transducer, through the acoustic coupling fluid, to a target stone in the body.

14. The method of claim 13 including moving acoustic coupling fluid from a reservoir toward the gel enclosure responsive to operation of the vacuum pump.

15. The method of claim 13 wherein the transmitting step is performed when a pressure in the gel enclosure is below atmospheric pressure.

16. The method of claim 13 including sensing for a presence of air bubbles in the gel enclosure.

17. The method of claim 16 including a step of stopping operation of the vacuum pump responsive to sensing an absence of air bubbles in the gel enclosure.

18. The method of claim 13 including forming the gel enclosure by contacting a first annular seal of a double sided cuff with the lithotripsy dry head shock wave transducer, and contacting a second annular seal of the double sided cuff with the body.

19. The method of claim 18 wherein the removing step includes moving air bubbles in the gel enclosure through a first port in an annular wall of the double sided cuff toward the vacuum pump.

20. The method of claim 19 including moving acoustic coupling fluid into the gel enclosure through a second port in the annular wall responsive to operation of the vacuum pump.
